(19) United States
(12) Patent Application Publication
(10) Pub. No.: US 2026/0209809 A1
(43) Pub. Date: Jul. 23, 2026

(54) METHOD AND DEVICE FOR MANUFACTURING COMPATIBLE SOLUTE

(71) Applicant: National University Corporation Tokyo University of Agriculture and Technology, Tokyo (JP)

(72) Inventors: Shohei Yasuda, Tokyo (JP); Akihiko Terada, Tokyo (JP)

(21) Appl. No.: 19/140,986

(22) PCT Filed: Nov. 17, 2023
(86) PCT No.: PCT/JP2023/041462

§ 371 Date: Jun. 18, 2025

(30) Foreign Application Priority Data
Dec. 20, 2022 (JP) 2022-203090

(51) Int. Cl. C12P 17/12; C12N 1/20; C12R 1/01

(52) U.S. Cl. CPC C12P 17/12; C12N 1/20; C12R 2001/01

(57) ABSTRACT

The method for manufacturing a compatible solute includes culturing a floc containing a halophilic microbial community using a culture solution containing salt to allow the halophilic microbial community to produce a compatible solute. The device for manufacturing a compatible solute includes a bioreactor configured to accommodate a culture solution containing salt and culture a floc containing a halophilic microbial community in the culture solution.

### TECHNICAL FIELD

The present invention relates to a method and a device for manufacturing a compatible solute.

Priority is claimed on Japanese Patent Application No. 2022-203090, filed Dec. 20, 2022, the content of which is incorporated herein by reference.

### BACKGROUND ART

It is known that halophilic microbial groups produce and accumulate compatible solutes to resist osmotic pressure in a high salt concentration environment. Ectoine, which is one of the compatible solutes, is commercially produced by Halomonas elongata, which is a halophilic Gram-negative bacterium, using glucose as a substrate (see, for example, Non-Patent Documents 1 and 2). In addition, as an improved method, studies have been in progress using halophilic methane-oxidizing bacteria (halophilic MOB) such as Methylomicrobium alcaliphilum 20Z, using methane as a substrate (see, for example, Non-Patent Documents 3 and 4). These bacteria are cultured in a salt-containing artificial medium to produce a compatible solute such as ectoine.

### CITATION LIST

### Non-Patent Documents

Non-Patent Document 1: Sauer T et al., “Bacterial milking: A novel bioprocess for production of compatible solutes.”, Biotechnol. Bioeng., Vol. 57, pp. 306-313, 1998.
Non-Patent Document 2: Pastor J. M et al., “Ectoines in cell stress protection: Uses and biotechnological production.”, Biotechnol. Adv., Vol. 28, pp. 782-801, 2010.
Non-Patent Document 3: Cantera S et al., “Ectoine bio-milking in methanotrophs: A step further towards methane-based bio-refineries into high added-value products.” Chem. Eng. J., Vol. 328, pp. 44-48, 2017.
Non-Patent Document 4: Cantera S et al., “Valorization of CH₄ emissions into high-added-value products: Assessing the production of ectoine coupled with CH₄ abatement.”, J. Environ. Manage., Vol. 182, pp. 160-165, 2016.

### SUMMARY OF INVENTION

### Technical Problem

In the method using Halomonas bacteria, it is necessary to purchase glucose used as a substrate, and there is an issue in that the high market price of ectoine is offset by the manufacturing cost because of an increase in cost.

In addition, since a single strain is used for both Halomonas bacteria and halophilic MOB, it is necessary to prevent the contamination with foreign microorganisms in the manufacturing process, and there is an issue that it takes time and effort to sterilize the manufacturing device and the substrate of the bacteria and then perform the supply, and the energy cost is high.

The present invention has been made in view of the above circumstances, and provides a method and a device for manufacturing a compatible solute, which are less likely to be affected by contamination with foreign microorganisms and do not require a sterilization treatment.

### Solution to Problem

That is, the present invention includes the following aspects.
(1) A method for manufacturing a compatible solute, including culturing a floc containing a halophilic microbial community using a culture solution containing salt to allow the halophilic microbial community to produce a compatible solute.
(2) The method for manufacturing a compatible solute according to (1), in which the halophilic microbial community is a microbial community including halophilic methane-oxidizing bacteria and halophilic methanol-utilizing bacteria.
(3) The method for manufacturing a compatible solute according to (1) or (2), in which the production is performed using a bioreactor capable of supplying a gas.
(4) The method for manufacturing a compatible solute according to (3), in which the bioreactor is a membrane biofilm reactor.
(5) The method for manufacturing a compatible solute according to any one of
(1) to (4), in which the culture solution is groundwater or an effluent, or a synthetic culture medium containing groundwater or an effluent.
(6) The method for manufacturing a compatible solute according to (5), in which the halophilic microbial community is derived from the groundwater or the effluent.
(7) The method for manufacturing a compatible solute according to any one of
(1) to (6), in which the floc is a biofilm.
(8) The method for manufacturing a compatible solute according to any one of
(1) to (7), in which the compatible solute is one or more selected from the group consisting of betaine, ectoine, hydroxyectoine, N-γ-acetyldiaminobutyric acid, N-ε-acetyl-β-lysine, β-glutamine, α-glucosyl glycerol, α-mannosyl glyceramide, trehalose, sucrose, N-α-carbamoyl-L-glutamine-1-amide, N-acetylglutaminyl glutamine amide, L-α-glutamic acid, β-glutamic acid, hydroxybutyric acid, poly-β-hydroxybutyric acid, α-glucosyl glyceric acid, α-mannosyl glyceric acid, α-diglycerol phosphate, di-myo-inositol-1,1′-phosphate, mannosyl-diinositol phosphate, cyclic 2,3-diphosphoglyceric acid, sulfotrehalose, and ceramide.
(9) The method for manufacturing a compatible solute according to any one of
(1) to (8), in which the compatible solute is ectoine or hydroxyectoine.
(10) The method for manufacturing a compatible solute according to any one of
(1) to (9), the method further including separating the compatible solute from the culture solution or the halophilic microbial community.
(11) A device for manufacturing a compatible solute, including a bioreactor configured to accommodate a culture solution containing salt and culture a floc containing a halophilic microbial community in the culture solution.
(12) The device for manufacturing a compatible solute according to (11), the device further including a separation unit configured to separate the compatible solute from the culture solution or the halophilic microbial community.

### Advantageous Effects of Invention

According to the manufacturing method and the manufacturing device of the above-described aspect, it is possible to provide a method and a device for manufacturing a compatible solute, which are less likely to be affected by the contamination with foreign microorganisms and do not require a sterilization treatment.

### BRIEF DESCRIPTION OF DRAWINGS

FIG. 1A A schematic configuration diagram of a membrane biofilm reactor used in the manufacturing method according to the present embodiment.

FIG. 1B A graph showing a substrate concentration profile in a membrane biofilm reactor used in the manufacturing method according to the present embodiment.

FIG. 2A A schematic configuration diagram showing an example of a device for manufacturing a compatible solute according to the present embodiment.

FIG. 2B A schematic configuration diagram showing an expanded example of a device for manufacturing a compatible solute according to the present embodiment.

FIG. 2C A schematic configuration diagram showing an expanded example of a device for manufacturing a compatible solute according to the present embodiment.

FIG. 3 A graph showing the relative abundance of microbial species by 16S rRNA gene phylogenetic analysis in Example 1.

FIG. 4 A graph showing the ectoine concentration in a biofilm in Example 1.

FIG. 5 A graph showing the yields of ectoine and hydroxyectoine on the 429th day from the start of culture in Example 1.

### DESCRIPTION OF EMBODIMENTS

Hereinafter, a method and a device for manufacturing a compatible solute according to the present embodiment will be described in detail with appropriate reference to the drawings.

It is noted that in each of the drawings used in the following description, the featured portions may be enlarged and shown to make the features of the present invention easy to understand, and a dimension ratio and the like of each component may be different from an actual state. In addition, materials, dimensions, and the like mentioned in the following description are examples, and the present invention is not limited thereto and can be implemented with appropriate modifications within the scope of the present invention.

### <<Compatible Solute>>

The compatible solute generally means a solute in an intracellular liquid that does not exhibit cytotoxicity even in a case of being accumulated at a high concentration and has the action of adjusting the osmotic pressure of cells and the action of stabilizing the structure and function of biopolymers such as proteins and biological membranes. The compatible solute is an organic compound having extremely high water solubility and a relatively low molecular weight. It is known that halophilic microbial groups produce and accumulate this compatible solutes to resist osmotic pressure in a high salt concentration environment.

The compatible solute manufactured by using the manufacturing method and the manufacturing device according to the present embodiment is not particularly limited as long as it is known as a compound generated and accumulated by the halophilic microorganisms to resist the osmotic pressure in a high salt concentration environment, and examples thereof include betaine, ectoine, hydroxyectoine, N-γ-acetyldiaminobutyric acid, N-ε-acetyl-β-lysine, β-glutamine, α-glucosyl glycerol, α-mannosyl glyceramide, trehalose, sucrose, N-α-carbamoyl-L-glutamine-1-amide, N-acetylglutaminyl glutamine amide, L-α-glutamic acid, β-glutamic acid, hydroxybutyric acid, poly-β-hydroxybutyric acid, α-glucosyl glyceric acid, α-mannosyl glyceric acid, α-diglycerol phosphate, di-myo-inositol-1,1′-phosphate, mannosyl-diinositol phosphate, cyclic 2,3-diphosphoglyceric acid, sulfotrehalose, ceramide, and the like. Among these, ectoine or hydroxyectoine is preferable.

### <<Method for Manufacturing Compatible Solute>>

The method for manufacturing a compatible solute according to the present embodiment (hereinafter, may be simply referred to as “the manufacturing method according to the present embodiment”) includes culturing a floc containing a halophilic microbial community using a culture solution containing salt to allow the halophilic microbial community to produce a compatible solute (hereinafter, may be referred to as “production step”).

In the conventional method for manufacturing a compatible solute, since a single halophilic bacterium is used, it is necessary to sterilize a manufacturing device, a substrate to be supplied, and the like.

On the other hand, in the manufacturing method according to the present embodiment, since the halophilic microbial community is significantly proliferated by using the floc containing the halophilic microbial community consisting of a plurality of types of bacteria, even in a case of contaminating with foreign microorganisms, the foreign microorganisms is hardly affected. Therefore, it is not necessary to sterilize the manufacturing device, the substrate to be supplied, and the like.

Hereinafter, the steps constituting the production method according to the present embodiment will be described in detail.

### <Production Step>

In the production step, the floc containing the halophilic microbial community is cultured using a culture solution containing salt to allow the halophilic microbial community to produce a compatible solute.

### [Floc]

The floc used in the production step contains a halophilic microbial community. That is, the floc contains a plurality of types of halophilic microorganisms.

In the present specification, the “floc” means an aggregate formed by accumulation of microorganisms. The shape of the floc is not particularly limited, and examples thereof include a spherical shape, a substantially spherical shape, and a membranous shape (film-like shape). Among these, the floc is preferably in film-like shape, that is, a biofilm. The biofilm means a biological film formed by microorganisms on a solid phase surface, and in the present specification, the biofilm is included in the floc. As described later, in a case where a device having a gas supply unit with a relatively large surface area, such as a membrane biofilm reactor, is used as a culture device for microorganisms, the biofilm is formed on the surface of the gas supply unit and the microorganisms constituting the biofilm are cultured while directly supplying a gas containing a substrate from the surface of the gas supply unit, whereby the compatible solute can be efficiently produced.

The type of halophilic microorganisms contained in the floc is not particularly limited as long as they are halophilic microorganisms and produce the above-described compatible solute, and examples thereof include halophilic methane-oxidizing bacteria (MOB) such as Methylomicrobium, halophilic methanol-utilizing bacteria (MAB) such as Methylophaga, halophilic anammox bacteria, and the like.

Among these, as the halophilic microorganisms contained in the floc, microorganisms producing compatible solutes using greenhouse gases such as carbon dioxide and methane as substrates are preferable, and the halophilic MOB and the halophilic MAB are more preferable. The halophilic MOB produces a metabolite containing methanol, formic acid, or the like in addition to the compatible solute by a methane oxidation reaction using methane as a substrate. Using methanol and the like as a substrate, the halophilic MAB produces a compatible solute. By utilizing the symbiotic relationship between these two types of microorganisms, it is possible to more efficiently produce a compatible solute. In addition, since the halophilic anammox bacteria can proliferate by using carbon dioxide, it is possible to produce a compatible solute while reducing greenhouse gases.

It is noted that, in the present specification, the “substrate” means a compound that serves as a main raw material (carbon source) of a compatible solute produced by an enzymatic reaction in a microorganism.

The halophilic microbial community may be formed by combining a plurality of the isolated strains of the above-mentioned halophilic microorganisms, may be derived from groundwater (including brine, hot spring water, and the like) or an effluent (including municipal effluent, industrial effluent, landfill leachate, and the like), or may be derived from activated sludge used for treating groundwater or effluent.

Among these, the halophilic microbial community is preferably derived from the groundwater or the effluent, and more preferably derived from the landfill leachate. Groundwater or effluent contains a nitrogen source such as ammonia, phosphorus, and trace metals in addition to various microorganisms. Therefore, in the manufacturing method according to the present embodiment, by adding salt to groundwater or effluent as necessary and using the groundwater or the effluent as a culture solution, it is possible to allow halophilic microorganisms to predominantly proliferate among microorganisms contained in the groundwater or the effluent while effectively utilizing nutrient components such as ammonia, phosphorus, and trace metals contained in the groundwater or the effluent, without using isolated strains of halophilic microorganisms, and the like.

### [Culture Solution]

The culture solution used in the production step is not particularly limited as long as it has a composition suitable for culturing halophilic microorganisms, for example, a composition containing a nitrogen source such as ammonia, phosphorus, trace metals, and the like, and for example, a synthetic culture medium shown in Examples described later, groundwater, effluent, or a mixture thereof can be used.

Among these, as the culture solution, the groundwater or the effluent, or the synthetic culture medium containing groundwater or effluent is preferable. As described above, groundwater or effluent also contains salts, a nitrogen source such as ammonia, phosphorus, and trace metals, in addition to various microorganisms. Therefore, in the manufacturing method according to the present embodiment, by using groundwater or effluent as the culture solution, it is possible to predominantly proliferate halophilic microorganisms among microorganisms contained in groundwater or effluent while effectively utilizing nutrient components such as salts, ammonia, phosphorus, and trace metals contained in groundwater or effluent, without using isolated strains of halophilic microorganisms, and the like. It is noted that salt, a nitrogen source such as ammonia, phosphorus, trace metals, or the like may be added to the groundwater or the effluent as necessary.

It is noted that the term “salt” in the present specification refers broadly to a compound formed of an anion and a cation, and particularly includes sodium chloride, magnesium chloride, magnesium sulfate, calcium sulfate, potassium chloride, and the like.

The concentration of the salts contained in the culture solution is not particularly limited as long as the concentration is suitable for the proliferation of the halophilic microorganism to be used, but for example, it can be set to 1 w/v % or more and 15 w/v % or less, or can be set to 2 w/v % or more and 12 w/v % or less.

### [Culture Conditions]

In the production step, the culture of the floc containing the halophilic microbial community may be batch culture, fed-batch culture (also referred to as semi-batch culture), or continuous culture. The culture method can be appropriately selected in consideration of the type of the halophilic microorganism to be used and the compound serving as the substrate, the location conditions of the equipment for performing the manufacturing method according to the present embodiment, and the like.

The culture device used in the production step can be appropriately selected depending on the type of the floc containing the halophilic microbial community. Among these, from the viewpoint of reducing environmental burden, in a case where a floc containing a halophilic microbial community that produces a compatible solute using a greenhouse gas such as carbon dioxide or methane as a substrate is used, it is preferable to use a bioreactor capable of supplying a gas.

The “bioreactor capable of supplying a gas” in the present specification may be any bioreactor as long as it can supply a gas in the bioreactor or in a culture solution in the bioreactor and can allow the gas to dissolve in the culture solution. Since the solubility of the gas is lowered in a culture solution containing salts, it is preferable to use a bioreactor configured to directly supply a gas containing a substrate from the surface of the gas supply unit to the microorganisms constituting the biofilm by having a gas supply unit having a relatively large surface area or a bioreactor configured to keep a gas containing a substrate dissolved in the culture solution by pressure. In these bioreactors, since aeration is not performed unlike the aeration type bioreactor in the related art, it is possible to supply a gas while suppressing the cost generated by aeration.

As the bioreactor having a gas supply unit with a relatively large surface area, it is preferable to use a gas permeable membrane type bioreactor that diffuses gas from the inside of a hollow membrane without bubbles.

As the gas, it is preferable to supply a gas containing a gas molecule that can be metabolized by a microorganisms, and for example, a gas containing a substrate, particularly a gas containing a gas molecule serving as a carbon source or oxygen can be supplied. In the present embodiment, it is particularly preferable to supply a gas containing a gas molecule serving as a carbon source, and as a result, a compatible solute can be produced using the gas as a carbon source. Examples of the gas molecule serving as a carbon source include carbon dioxide, methane gas, and the like, and as a gas including such a gas molecule, natural gas, biogas generated by fermentation, and the like can be used.

For example, in a case where a halophilic microbial community containing halophilic MOB and halophilic MAB is used, since the methane gas serving as a substrate has a property of being difficult to dissolve in water, it is preferable to use a bioreactor having a gas supply unit with a relatively large surface area as the culture device. It is preferable that the gas supply unit be formed of a flat membrane-like film, a spiral-type film, or a hollowfiber membrane. Among these, a membrane biofilm reactor (MBfR) is particularly preferably used as such a bioreactor.

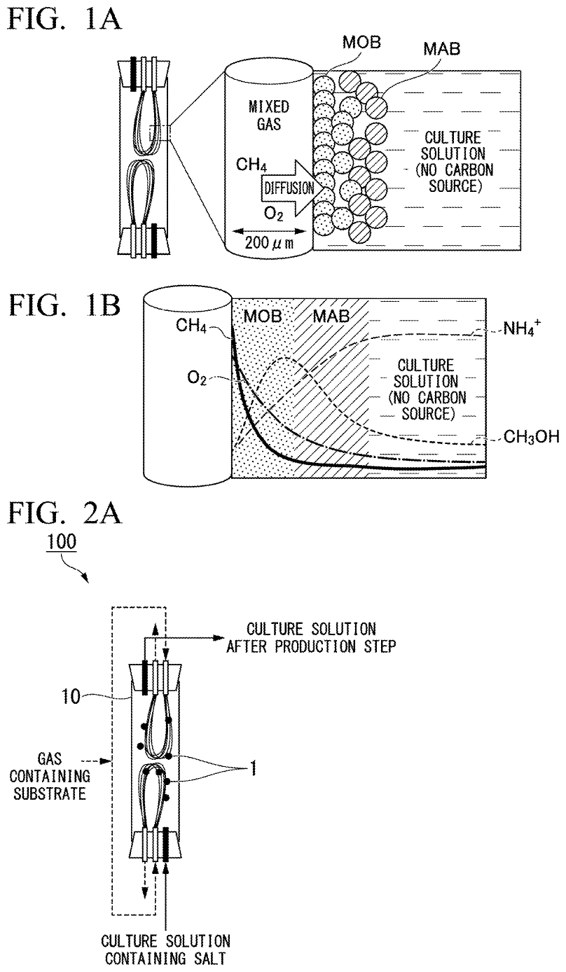

FIG. 1A is a schematic configuration diagram of a membrane biofilm reactor used in the manufacturing method according to the present embodiment, and FIG. 1B is a graph showing a substrate concentration profile in the membrane biofilm reactor.

As shown in FIG. 1A, in the MBfR, oxygen or methane, which is a poorly soluble gas, can be efficiently dissolved or diffused in the culture solution without generating bubbles from the inside of the membrane. In addition, as shown in FIGS. 1A and 1B, the halophilic MOB and the halophilic MAB are accumulated on the surface of the membrane to form a biofilm containing these halophilic microbial community, whereby methane and oxygen can be directly supplied to the halophilic MOB from the surface of the membrane, and on the other hand, a nitrogen source contained in the culture solution is used as a nutrient component. As a result, halophilic MOB proliferates efficiently, and a suitable compatible solute can be produced with a good yield. Furthermore, the halophilic MAB is also accumulated together with the halophilic MOB by methanol produced by the halophilic MOB, whereby the production of a compatible solute by the halophilic MAB can also be expected.

In a case of using a halophilic microbial community containing halophilic MOB and halophilic MAB, the concentration of methane gas serving as a substrate can be set to, for example, 1.0% by volume (v/v %) or more and 50.0 v/v % or less, 5.0 v/v % or more and 25.0 v/v % or less, or 18.0 v/v % or more and 22.0 v/v % or less, with respect to the total gas volume. In addition, the concentration of oxygen can be set to, for example, 1.0 v/v % or more and 50.0 v/v % or less, 13.0 v/v % or more and 21.0 v/v % or less, or 16.5 v/v % or more and 17.0 v/v % or less, with respect to the total gas volume.

In addition, for example, in a case of using a halophilic microbial community containing halophilic anammox bacteria, since carbon dioxide serving as a substrate has a property of being easily dissolved in water, it is preferable to use a bioreactor configured to keep a gas containing a substrate dissolved in the culture solution by pressure. It is noted that, in a case of using a halophilic microbial community containing halophilic anammox bacteria, the concentration of carbon dioxide and the gas flow rate can be appropriately adjusted as long as gas partial pressure of the supplied carbon dioxide satisfies the stoichiometric ratio based on the kinetic formula described in Reference Document 1 (Lotti T. et al., “Physiological and kinetic characterization of a suspended cell anammox culture.”, WATER RESEARCH, Vol. 60, pp. 1-14, 2014.).

In the production step, the culture temperature is not particularly limited as long as it is a temperature suitable for the halophilic microorganism to be used, and for example, it can be set to 10° C. or higher and 60° C. or lower, and it can be set to 20° C. or higher and 40° C. or lower.

In the production step, the culture period is only required to be a period sufficient for the production of the compatible solute by the halophilic microorganism, and can be appropriately set.

The manufacturing method according to the present embodiment can further include other steps in addition to the above-described production step.

Examples of the other steps include a floc forming step, a separation step, a purification step, a desalting step, and the like.

### <Floc Forming Step>

The floc forming step is performed before the above-described production step, and a plurality of types of microorganisms including halophilic microorganisms are cultured using a culture solution containing salt to form a floc containing a halophilic microbial community.

The floc forming step and the above-described production step can be continuously performed in the same culture device.

Examples of the culture solution, the halophilic microorganism, and the culture device used in the floc forming step include the same ones as exemplified in the above-described production step.

The culture temperature in the floc forming step is the same as the conditions exemplified in the above-described production step.

The culture period in the floc forming step is not particularly limited as long as the halophilic microbial community can form a floc having a sufficient size, and for example, it can be set to 1 day or more, 5 days or more, or 14 days or more.

### <Separation Step>

The separation step is performed after the above-described production step, and the compatible solute is separated from the culture solution or the halophilic microbial community.

The compatible solute is accumulated inside the halophilic microorganisms and mainly plays a role of adjusting the osmotic pressure, but may be partially released from the inside of the halophilic microorganisms into the culture solution. Therefore, the compatible solute is present inside the halophilic microorganism and in the culture solution. Therefore, the compatible solute can be isolated by separating the compatible solute from the inside of the halophilic microorganisms and the culture solution.

Examples of the method of separating the compatible solute from the inside of the halophilic microorganisms include a method known as “biomilking” in which only the culture solution is discharged while only the floc containing a halophilic microbial community is held in a culture device, and the culture solution is replaced with a culture solution in which the salt concentration is decreased, thereby lowering the osmotic pressure and releasing the compatible solute from the inside of the microbial body. Alternatively, for example, after the floc containing the halophilic microbial community is recovered, a solvent such as ethanol is used to extract a compatible solute from the halophilic microorganisms into the solvent.

Examples of the method of separating the compatible solute from the culture solution include, but are not limited to, centrifugation, membrane separation (including microfiltration, ultrafiltration, nanofiltration, reverse osmosis, dialysis, electrodialysis, and the like), adsorption separation with an ion exchange resin or the like, distillation, solvent extraction, and the like. Depending on the type of the compatible solute, an appropriate method can be selected from the above-described separation methods, and the separation can be performed alone or a combination of two or more methods can be used.

In addition, in the separation step, in a case where the compatible solute is separated by biomilking, only the culture solution in which the salt concentration is decreased is discharged while only the floc containing the halophilic microbial community is held in the culture device, and the culture solution in which the salt concentration is increased is supplied to the inside of the device again, whereby the compatible solute can be produced and accumulated in the floc of the halophilic microbial community held in the device, and this cycle can be repeated.

### <Purification Step>

The purification step is performed after the separation step, and in the purification step, the compatible solute separated from the culture solution or the halophilic microbial community is purified.

The purification step may or may not be performed depending on the use application of the compatible solute, but in a case where the purification is performed, a compatible solute having a higher purity can be obtained.

Examples of the purification method include concentration, crystallization, reprecipitation, column chromatography, solvent extraction, stirring and washing of crystals with a solvent, and the like, but the purification method is not limited thereto. Depending on the type of the compatible solute, an appropriate method can be selected from the above-described purification methods, and the purification can be performed alone or a combination of two or more methods can be used.

### <Desalting Step>

In the desalting step, salts are extracted from a culture solution discharged after the production step, a waste liquid discharged after the separation step, or groundwater or effluent, and the salts are reused for adjusting the salt concentration of the culture solution used in the production step. The culture solution or the waste liquid from which the salt has been removed can be reused as it is, or can be reused after adding components such as salt taken out in the desalting step or new salt. Alternatively, the remaining solution may be discharged to the outside of the system.

By performing the desalting step, it is possible to perform the production step without adding new salt from the outside, and it is possible to further reduce the manufacturing cost. In addition, since the waste liquid containing a high concentration of salt is not discharged outside the system, it is possible to reduce the environmental burden.

The desalting step may be performed before, after, or at the same time as the above-described production step. The timing at which the desalting step is performed can be appropriately selected depending on the type of the solution to be subjected to desalting.

Examples of the desalting method include, but are not limited to, membrane separation (including microfiltration, ultrafiltration, nanofiltration, reverse osmosis, dialysis, electrodialysis, and the like), adsorption with an ion exchange resin and the like, distillation, and solvent extraction, and the like.

### <<Device for Manufacturing Compatible Solute>>

A device for manufacturing a compatible solute according to the present embodiment (hereinafter, may be simply referred to as a “manufacturing device according to the present embodiment”) includes a bioreactor configured to accommodate a culture solution containing salt and culture a floc containing a halophilic microbial community in the culture solution.

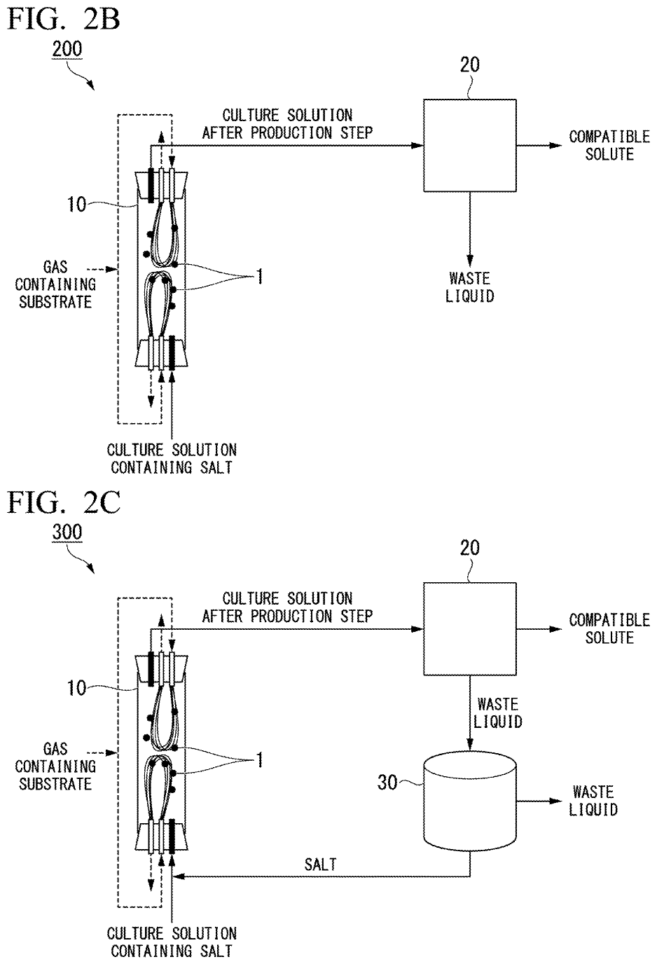

FIG. 2A is a schematic configuration diagram showing an example of a manufacturing device according to the present embodiment.

A device for manufacturing a compatible solute 100 shown in FIG. 2A include a bioreactor 10. The bioreactor 10 is configured to accommodate a culture solution containing salt and culture a floc 1 containing a halophilic microbial community in the culture solution.

### <Bioreactor>

In FIG. 2A, an aspect in which the bioreactor 10 is an MBfR is exemplified, but the bioreactor 10 can be appropriately selected depending on the type of the floc containing the halophilic microbial community. Among these, from the viewpoint of reducing environmental burden, in a case where a floc containing a halophilic microbial community that produces a compatible solute using a greenhouse gas such as carbon dioxide or methane as a substrate is used, it is preferable to use a bioreactor capable of supplying a gas. Examples of the bioreactor capable of supplying a gas include the same ones as those exemplified in the above-described method for manufacturing a compatible solute.

FIG. 2B is a schematic configuration diagram showing a modification example of the manufacturing device according to the present embodiment.

The device for manufacturing a compatible solute 200 shown in FIG. 2B is different from the device for manufacturing a compatible solute 100 shown in FIG. 2A in that the separation unit 20 is further provided. It is noted that, in FIG. 2B and the subsequent drawings, the same components as shown in FIG. 2A will be denoted by the same reference numerals, and the description thereof will not be repeated.

### <Separation Unit>

The separation unit 20 is configured to separate the compatible solute from the culture solution or the halophilic microbial community.

In a case where the culture solution fed to the separation unit 20 contains a floc containing a halophilic microbial community, the separation unit is configured with a first separation unit configured to separate the culture solution from the floc containing a halophilic microbial community, a second separation unit configured to separate the compatible solute from the culture solution, and a third separation unit configured to separate the compatible solute from the floc containing a halophilic microbial community.

As the first separation unit, for example, a solid-liquid separation device, a centrifugation device, a filtration membrane, or the like can be used, but the present invention is not limited thereto. In addition, these may be used alone or a combination of two or more types may be used.

As the second separation unit, for example, a culture device that is usually used for biomilking, a reaction tank that is used for solvent extraction, or the like can be used, but the present invention is not limited thereto. In addition, these may be used alone or a combination of two or more types may be used.

As the third separation unit, for example, a membrane separation device (including a microfiltration device, an ultrafiltration device, a nanofiltration device, a reverse osmosis membrane filtration device, a dialysis device, an electrodialysis device, and the like), a column chromatogram including an ion exchange resin and the like, a distillation column, a reaction tank that is used for solvent extraction, and the like can be used, but the present invention is not limited thereto. In addition, these may be used alone or a combination of two or more types may be used.

In addition, in a case where the culture solution fed to the separation unit 20 does not contain the floc containing the halophilic microbial community, as the separation unit, one similar to the exemplified third separation unit can be used.

FIG. 2C is a schematic configuration diagram showing a modification example of the manufacturing device according to the present embodiment.

The device for manufacturing a compatible solute 300 shown in FIG. 2C is different from the device for manufacturing a compatible solute 200 shown in FIG. 2B in that the desalting unit 30 is further provided.

### <Desalting Unit>

The desalting unit 30 is configured to extract salt from the culture solution discharged from the bioreactor 10, the waste liquid discharged from the separation unit 20, or groundwater or effluent, that is, to perform desalting treatment. It is noted that, in FIG. 2C, an aspect in which only the waste liquid discharged from the separation unit 20 is fed has been illustrated, but the culture solution discharged from the bioreactor 10, groundwater, or the effluent can also be fed to the desalting unit 30.

As the desalting unit 30, for example, a membrane separation device (including a microfiltration device, an ultrafiltration device, a nanofiltration device, a reverse osmosis membrane filtration device, a dialysis device, an electrodialysis device, and the like), a column chromatogram including an ion exchange resin and the like, a distillation column, and the like can be used, but the present invention is not limited thereto. In addition, these may be used alone or a combination of two or more types may be used.

The manufacturing device of the present embodiment is not limited to the manufacturing device shown in FIGS. 2A to 2C and a part of the configurations shown in FIGS. 2A to 2C may be changed or deleted, or other configurations may be further added to those described so far, in a range in which the effects of the present invention are not impaired.

For example, in the manufacturing device shown in FIGS. 2A to 2C, a microbial treatment tank may be further provided in a rear stage of the bioreactor 10 shown in FIG. 2A, a rear stage of the separation unit 20 shown in FIG. 2B, or a rear stage of the separation unit 20 shown in FIG. 2C, and a front stage of the desalting unit 30.

In the microbial treatment tank, the organic substances or ammonia contained in the culture solution discharged from the bioreactor 10 or the waste liquid discharged from the separation unit 20 is treated by microorganisms. As a result, the amount of organic substances and ammonia contained in the waste liquid finally discharged into rivers or sewage can be reduced, and the environmental burden can be reduced.

### EXAMPLES

Hereinafter, the present invention will be described with reference to Examples, but the present invention is not limited to the following Examples.

### Example 1

### (Examination on Lab Scale)

### 1. Preparation of Culture Medium

A non-sterile culture medium (salt concentration: 3 w/v %) was prepared to have the composition shown in the following table. It is noted that a 1 M stock solution of NaHCO₃ and a 1 M stock solution of Na₂CO₃ were each added in the amounts shown in the following table.

TABLE 1
Composition of culture medium

| | | |
|---|---|---|
| NaCl | 30.00 | g |
| MgSO₄•7H₂O | 0.20 | g |
| CaCl₂•2H₂O | 0.02 | g |
| NH₄Cl | 0.53 | g |
| Trace elements (see table described below) | 1.00 | mL |
| Distilled water | 1,000.00 | mL |
| NaHCO₃ (1M) | 50.0 | mL/L |
| Na₂CO₃ (1M) | 5.0 | mL/L |
| Phosphate buffer (see table described below) | 20.0 | mL/L |

| | |
|---|---|
| Final pH | 8.5 to 9.0 |

TABLE 2
Composition of trace elements

| | | |
|---|---|---|
| EDTA | 5.00 | g |
| CuCl₂•5H₂O | 0.10 | g |
| FeSO₄•7H₂O | 2.00 | g |
| ZnSO₄•7H₂O | 0.10 | g |
| NiCl₂•6H₂O | 0.02 | g |
| CoCl₂•6H₂O | 0.20 | g |
| Na₂MoO₄ | 0.03 | g |
| MnCl₂•4H₂O | 0.03 | g |
| H₃BO₃ | 0.03 | g |
| Distilled water | 1,000.00 | mL |

TABLE 3
Composition of phosphate buffer

| | | |
|---|---|---|
| KH₂PO₄ | 14.0 | g |
| Na₂HPO₄•12H₂O | 30.0 | g |
| Distilled water | 1,000.0 | mL |

### 2. Formation of Biofilm

1 mL of activated sludge from a landfill leachate treatment plant was added to 0.3 L of the non-sterile culture medium prepared in “1.” above, and the culture was performed at 30° C. while supplying a substrate gas containing methane and oxygen (methane concentration of 20.0 v/v % and oxygen concentration of 16.8 v/v % in substrate gas) using a hollow fiber membrane biofilm reactor (MBfR) as a liquid culture device.

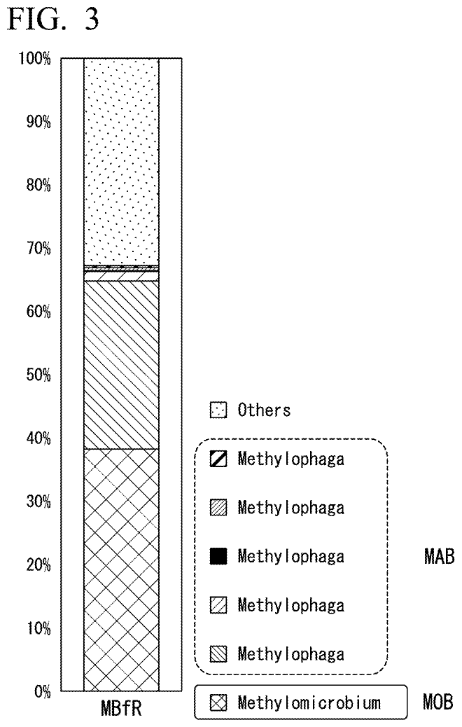

On the fifth day from the start of the culture, the proliferation of halophilic MOB was observed on the membrane surface of MBfR, and thereafter, a biofilm centered on halophilic MOB and MAB was formed. The relative abundance of the microbial species on the 429th day from the start of the culture was confirmed using a method of 16S rRNA gene phylogenetic analysis. The results are shown in FIG. 3.

As shown in FIG. 3, it was confirmed that Methylomicrobium, known as a halophilic MOB, accounted for 29%, and Methylophaga, belonging to MAB, accounted for 20%, and MOB and MAB accounted for 49% of the entire amount. It is considered that both the MAB and the MOB proliferated predominantly because the carbon source used for the proliferation of the microbial cells was only methane supplied as a substrate gas, the methane was utilized by the MOB, and the methanol produced and released by the metabolism was utilized by the MAB.

In addition, it is presumed that the reason why the relative abundance of the microbial species as shown in FIG. 3 was obtained is that halophilic microbial groups such as halophilic MOB and MAB always flowed into the activated sludge of the landfill leachate treatment plant from the leachate and were predominantly present in the activated sludge. It is considered that the main factor constituting the microorganism groups in the activated sludge is the composition of the effluent or groundwater that is the inflow water, and halophilic microorganisms originally present in the effluent are predominantly proliferating.

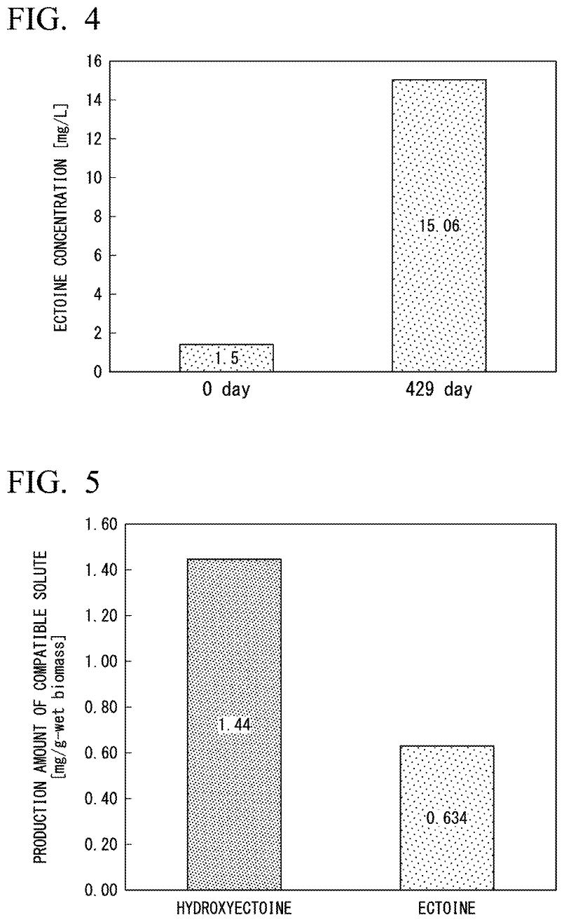

Next, FIG. 4 shows the results of measuring the ectoine concentration (N=2) accumulated in the biofilm.

As shown in FIG. 4, the accumulation amount increased from 1.5 mg/L at the start of the culture, and increased to 15.06 mg/L on the 429th day from the start of the culture. Therefore, it was confirmed that the accumulation of MOB and MAB progressed, the relative abundance thereof increased, and the accumulation amount of ectoine increased.

In addition, the production amounts of ectoine and hydroxyectoine per unit microbial body on the 429th day from the start of the culture are shown in the following table and FIG. 5.

TABLE 4

| | | Hydroxyectoine | | Ectoine | |
|---|---|---|---|---|---|
| | Concentration | | Yield | | Yield |
| | of bacterial | Concentration | (mg/g-wet | Concentration | (mg/g-wet |
| Sample | cells (g/mL) | (mg/L) | biomass) | (mg/L) | biomass) |
| 1 | 0.0346 | 47.2 | 1.36 | 16.2 | 0.468 |
| 2 | 0.0174 | 26.5 | 1.52 | 13.9 | 0.799 |
| Average | 0.0260 | 36.8 | 1.44 | 15.1 | 0.634 |
| value | | | | | |

As shown in the above table and FIG. 5, the production amounts of ectoine and hydroxyectoine were 0.634 mg/g-wet biomass and 1.44 mg/g-wet biomass.

From the above results, it was clarified that, by using the manufacturing method and the manufacturing device according to the present embodiment, compatible solutes such as ectoine and hydroxyectoine could be efficiently manufactured without performing the sterilization treatment.

### INDUSTRIAL APPLICABILITY

According to the manufacturing method and the manufacturing device according to the present embodiment, it is possible to provide a method and a device for manufacturing a compatible solute, which are less likely to be affected by the contamination with foreign microorganisms and do not require a sterilization treatment.

### REFERENCE SIGNS LIST

1 Floc containing halophilic microbial community
10 Bioreactor
20 Separation unit
30 Desalting unit
100, 200, 300 Device for manufacturing compatible solute

What is claimed is:

1. A method for manufacturing a compatible solute, comprising:
culturing a plurality of types of microorganisms including a halophilic microorganism derived from groundwater or an effluent, or an activated sludge used for treatment of the groundwater or the effluent, using a culture solution containing salt, to form a floc containing a halophilic microbial community; and
culturing the floc using the culture solution containing salt to allow the halophilic microbial community to produce a compatible solute,
wherein the floc is formed and the compatible solute is produced, using a membrane biofilm reactor capable of supplying a gas containing methane and oxygen, while supplying the gas to the microorganisms, and
the culture solution and the membrane biofilm reactor are non-sterile.

2. The method for manufacturing a compatible solute according to claim 1,
wherein the halophilic microbial community is a microbial community including halophilic methane-oxidizing bacteria and halophilic methanol-utilizing bacteria.

3. (canceled)

4. (canceled)

5. The method for manufacturing a compatible solute according to claim 1,
wherein the culture solution is groundwater or an effluent, or a synthetic culture medium containing groundwater or an effluent.

6. (canceled)

7. The method for manufacturing a compatible solute according to any one of claims 1, 2, and 5,
wherein the floc is a biofilm.

8. The method for manufacturing a compatible solute according to any one of claims 1, 2, and 5,
wherein the compatible solute is one or more selected from the group consisting of betaine, ectoine, hydroxyectoine, N-γ-acetyldiaminobutyric acid, N-ϵ-acetyl-β-lysine, β-glutamine, α-glucosyl glycerol, α-mannosyl glyceramide, trehalose, sucrose, N-α-carbamoyl-L-glutamine-1-amide, N-acetylglutaminyl glutamine amide, L-α-glutamic acid, β-glutamic acid, hydroxybutyric acid, poly-β-hydroxybutyric acid, α-glucosyl glyceric acid, α-mannosyl glyceric acid, α-diglycerol phosphate, di-myo-inositol-1,1′-phosphate, mannosyl-diinositol phosphate, cyclic 2,3-diphosphoglyceric acid, sulfotrehalose, and ceramide.

9. The method for manufacturing a compatible solute according to any one of claims 1, 2, and 5,
wherein the compatible solute is ectoine or hydroxyectoine.

10. The method for manufacturing a compatible solute according to any one of claims 1, 2, and 5, the method further comprising:
separating the compatible solute from the culture solution or the halophilic microbial community.

11. A device for manufacturing a compatible solute, comprising:
a non-sterile membrane biofilm reactor configured to accommodate a culture solution containing salt, supply a gas containing methane and oxygen to the culture solution, and culture a floc containing a halophilic microbial community in the culture solution; and
a separation unit configured to separate the compatible solute from the culture solution or the halophilic microbial community.

12. The device for manufacturing a compatible solute according to claim 11,
wherein the separation unit is configured with a first separation unit configured to separate the culture solution from the floc, a second separation unit configured to separate a compatible solute from the culture solution, and a third separation unit configured to separate a compatible solute from the floc.
